(19) United States
(12) Patent Application Publication
(10) Pub. No.: US 2024/0068731 A1
(43) Pub. Date: Feb. 29, 2024

(54) REFRIGERATOR APPLIANCE WITH IMAGE-ASSISTED ODOR CONTROL

(71) Applicant: Haier US Appliance Solutions, Inc., Wilmington, DE (US)

(72) Inventors: Vineeth Vijayan, Louisville, KY (US); Stephanos Kyriacou, Louisville, KY (US)

(21) Appl. No.: 17/898,613

(22) Filed: Aug. 30, 2022

(51) Int. Cl. F25D 17/04; A61L 9/01; F25D 17/06; F25D 29/00; G06V 20/52; G06V 20/68; H04N 7/18

(52) U.S. Cl. CPC F25D 17/045; A61L 9/01; F25D 17/062; F25D 29/008; G06V 20/52; G06V 20/68; H04N 7/18; F25D 31/005

(57) ABSTRACT

A refrigerator appliance includes a cabinet defining a food storage chamber with a drawer slidably mounted within the food storage chamber therein and a camera assembly positioned and configured with at least a portion of the food storage chamber within a field of view of the camera assembly. A method of operating the refrigerator appliance includes obtaining an image using the camera assembly. The method also includes analyzing the image and identifying, based on the analysis of the image, a potential odor source in the food storage chamber. The method further includes initiating an enhanced odor control mode in response to the identified potential odor source.

### FIELD OF THE INVENTION

The present subject matter relates generally to refrigerator appliances, and more particularly to systems and methods for reducing or eliminating perceptible odors in such refrigerator appliances, e.g., which may be generated by items stored therein.

### BACKGROUND OF THE INVENTION

Refrigerator appliances generally include a cabinet that defines a chilled chamber. A wide variety of food items may be stored within the chilled chamber. The low temperature of the chilled chamber relative to ambient atmosphere assists with increasing a shelf life of the food items stored within the chilled chamber.

Various food items, such as produce items (e.g., fruits and vegetables), meats, cheeses, herbs (e.g., onion or garlic), stored in a refrigerator appliance may emit user-perceptible odors. In particular, some such food items may emit user-perceptible odors, or increasing levels of user-perceptible odors, as they go through various physical and chemical changes over time, e.g., ripening fruit, aging meat or cheese, etc. The presence of such user-perceptible odors is generally not desired as the odors may be unpleasant or annoying to at least some users.

Accordingly, a refrigerator appliance with systems for improved odor management, e.g., reducing or eliminating user-perceptible odors within the refrigerator appliance, would be useful.

### BRIEF DESCRIPTION OF THE INVENTION

Aspects and advantages of the invention will be set forth in part in the following description, or may be apparent from the description, or may be learned through practice of the invention.

In an exemplary embodiment, a method of operating a refrigerator appliance is provided. The refrigerator appliance includes a cabinet defining a food storage chamber therein and a camera assembly positioned and configured with at least a portion of the food storage chamber within a field of view of the camera assembly. The method includes obtaining an image using the camera assembly. The method also includes analyzing the image and identifying, based on the analysis of the image, a potential odor source in the food storage chamber. The method further includes initiating an enhanced odor control mode in response to the identified potential odor source.

In another exemplary embodiment, a refrigerator appliance is provided. The refrigerator appliance includes a cabinet defining a food storage chamber therein. The refrigerator appliance also includes a camera assembly positioned and configured with at least a portion of the food storage chamber within a field of view of the camera assembly. The refrigerator appliance further includes a controller. The controller is configured for obtaining an image using the camera assembly. The controller is also configured for analyzing the image and identifying, based on the analysis of the image, a potential odor source in the food storage chamber. The controller is further configured for initiating an enhanced odor control mode in response to the identified potential odor source.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a front view of a refrigerator appliance according to an exemplary embodiment of the present subject matter.

FIG. 2 provides a perspective view of the refrigerator appliance of FIG. 1.

FIG. 3 provides a front view of the refrigerator appliance of FIG. 1 with doors thereof in an open position.

FIG. 4 provides a schematic illustration of an example sealed cooling system as may be used with a refrigerator appliance in one or more exemplary embodiments of the present subject matter.

FIG. 5 is a perspective view of a portion of a sub-assembly for a refrigerator appliance according to one or more exemplary embodiments of the present subject matter which may be incorporated into a refrigerator appliance such as the exemplary refrigerator appliance of FIG. 1.

FIG. 6 is a close-up view of an air grille in the sub-assembly of FIG. 5.

FIG. 7 is a view of the air grille of FIG. 6 with a filter cover thereof in an open position.

FIG. 8 provides a flow diagram of an exemplary method for operating a refrigerator appliance according to one or more exemplary embodiments of the present subject matter.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

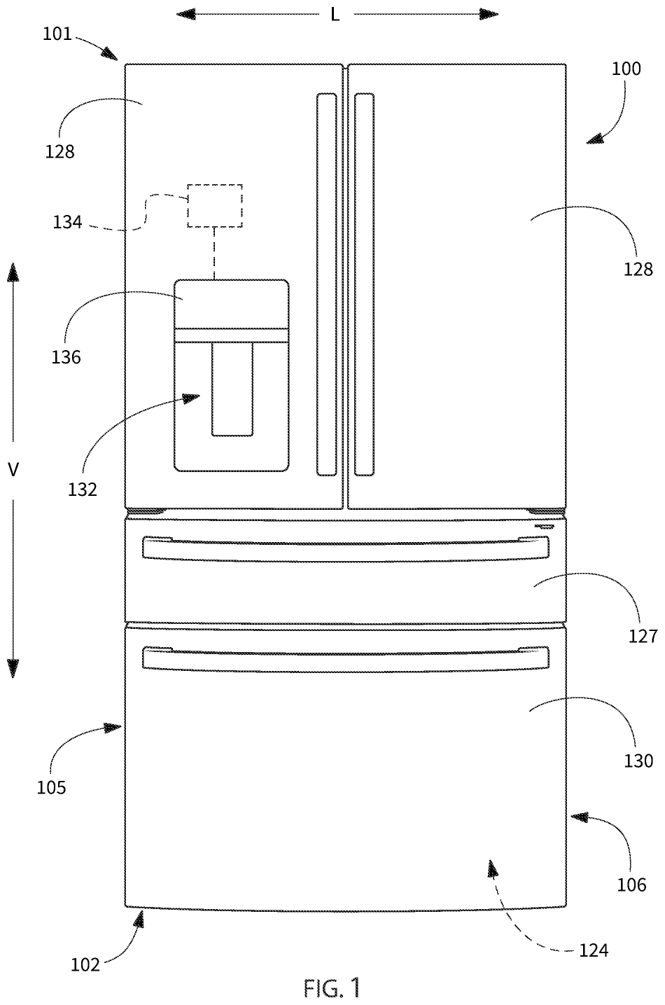

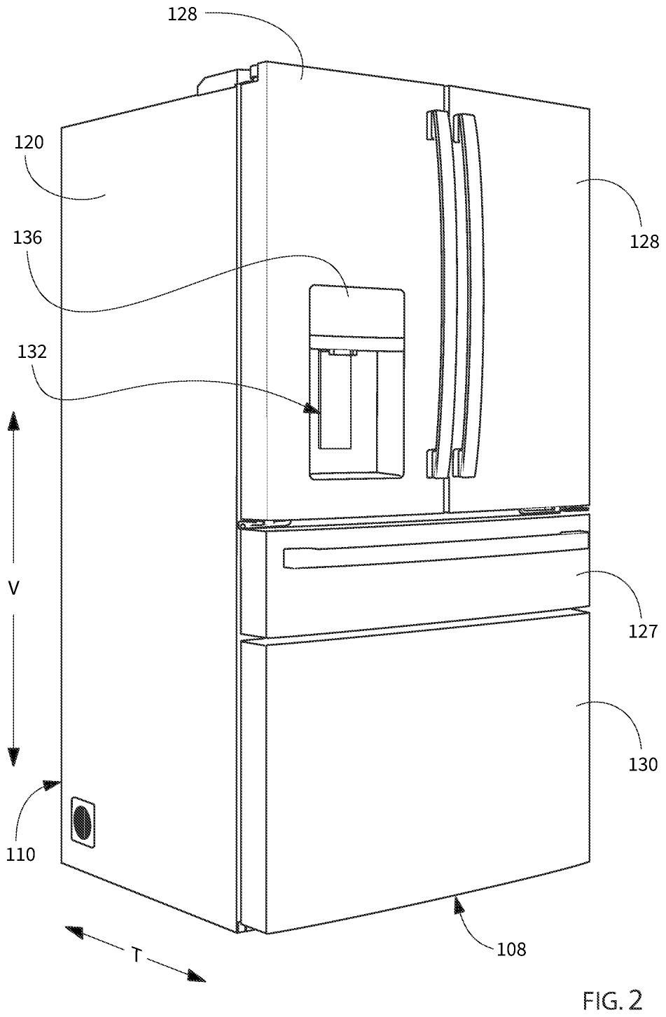

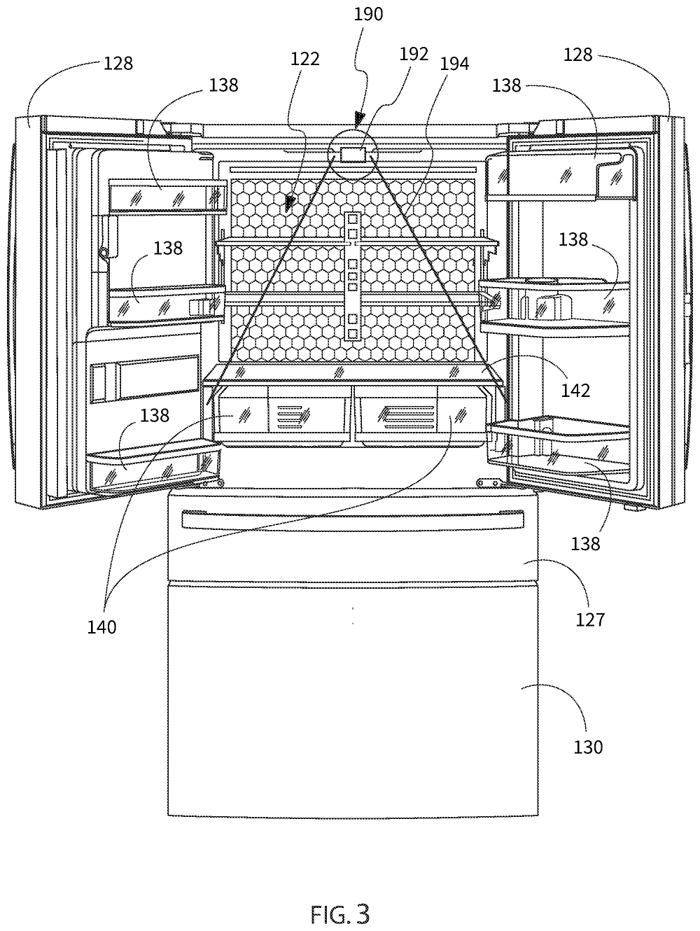

FIG. 1 is a front view of an exemplary embodiment of a refrigerator appliance 100. FIG. 2 is a perspective view of the refrigerator appliance 100. FIG. 3 is a front view of the refrigerator appliance 100 with fresh food doors 128 thereof in an open position. Refrigerator appliance 100 extends between a top 101 and a bottom 102 along a vertical direction V. Refrigerator appliance 100 also extends between a first side 105 and a second side 106 along a lateral direction L. As shown in FIG. 2, a transverse direction T may additionally be defined perpendicular to the vertical and lateral directions V and L. Refrigerator appliance 100 extends along the transverse direction T between a front portion 108 and a back portion 110.

Refrigerator appliance 100 includes a cabinet or housing 120 defining an upper fresh food chamber 122 (FIG. 3) and a lower freezer chamber or frozen food storage chamber 124 arranged below the fresh food chamber 122 along the vertical direction V. In some embodiments, an auxiliary food storage chamber (not shown) may be positioned between the fresh food storage chamber 122 and the frozen food storage chamber 124, e.g., along the vertical direction V. Because the frozen food storage chamber 124 is positioned below the fresh food storage chamber 122, refrigerator appliance 100 is generally referred to as a bottom mount refrigerator. In the exemplary embodiment, housing 120 also defines a mechanical compartment (not shown) for receipt of a sealed cooling system (not shown). Using the teachings disclosed herein, one of skill in the art will understand that the present invention can be used with other types of refrigerators (e.g., side-by-sides) as well. Consequently, the description set forth herein is for illustrative purposes only and is not intended to limit the invention in any aspect.

Refrigerator doors 128 are each rotatably hinged to an edge of housing 120 for accessing fresh food chamber 122. It should be noted that while two doors 128 in a “French door” configuration are illustrated, any suitable arrangement of doors utilizing one, two or more doors is within the scope and spirit of the present disclosure. A freezer door 130 is arranged below refrigerator doors 128 for accessing freezer chamber 124. In the exemplary embodiment, freezer door 130 is coupled to a freezer drawer (not shown) slidably mounted within freezer chamber 124. An auxiliary door 127 may be coupled to an auxiliary drawer (not shown) which is slidably mounted within the auxiliary chamber (not shown).

Operation of the refrigerator appliance 100 can be regulated by a controller 134 that is operatively coupled to a user interface panel 136. User interface panel 136 provides selections for user manipulation of the operation of refrigerator appliance 100 to modify environmental conditions therein, such as temperature selections, etc. In some embodiments, user interface panel 136 may be proximate to a dispenser assembly 132. Panel 136 provides selections for user manipulation of the operation of refrigerator appliance 100 such as, e.g., temperature selections, selection of automatic or manual override humidity control (as described in more detail below), etc. In response to user manipulation of the user interface panel 136, the controller 134 operates various components of the refrigerator appliance 100. Operation of the refrigerator appliance 100 can be regulated by the controller 134, e.g., controller 134 may regulate operation of various components of the refrigerator appliance 100 in response to programming and/or user manipulation of the user interface panel 136.

The controller 134 may include a memory and one or more microprocessors, CPUs or the like, such as general or special purpose microprocessors operable to execute programming instructions or micro-control code associated with operation of refrigerator appliance 100. The memory may represent random access memory such as DRAM, or read only memory such as ROM or FLASH. In one embodiment, the processor executes programming instructions stored in memory. The memory may be a separate component from the processor or may be included onboard within the processor. It should be noted that controllers 134 as disclosed herein are capable of and may be operable to perform any methods and associated method steps as disclosed herein.

The controller 134 may be positioned in a variety of locations throughout refrigerator appliance 100. In the illustrated embodiment, the controller 134 may be located within the door 128. In such an embodiment, input/output (“I/O”) signals may be routed between the controller and various operational components of refrigerator appliance 100. In one embodiment, the user interface panel 136 may represent a general purpose I/O (“GPIO”) device or functional block. In one embodiment, the user interface 136 may include input components, such as one or more of a variety of electrical, mechanical or electro-mechanical input devices including rotary dials, push buttons, and touch pads. The user interface 136 may include a display component, such as a digital or analog display device designed to provide operational feedback to a user. For example, the user interface 136 may include a touchscreen providing both input and display functionality. The user interface 136 may be in communication with the controller via one or more signal lines or shared communication busses.

As may be seen in FIG. 3, a plurality of food storage elements, such as bins 138, shelves 142, and drawers 140 are disposed within the fresh food storage chamber 122. As will be described in more detail below, the drawers 140 may be configured for storing produce, such as fruits and vegetables, and, in particular, the refrigerator appliance may be operable to provide, and configured for providing, improved shelf life of the produce stored therein. Thus, the drawers 140 may also be referred to as produce drawers 140 or as a vegetable drawer 140 and a fruit drawer 140. For example, in some embodiments, the refrigerator appliance may include two drawers 140, e.g., as illustrated in FIG. 3.

Also as may be seen in FIG. 3, refrigerator appliance 100 may further include an imaging system that is generally configured to monitor one or more chambers of refrigerator appliance 100. More specifically, as described in more detail below, the imaging system may include one or more cameras 192. As shown schematically in FIG. 3, the imaging system may include a camera assembly 190 that is generally positioned and configured for obtaining images of refrigerator appliance 100 during operation. Specifically, according to the illustrated embodiment, camera assembly 190 includes one or more cameras 192 that are mounted to cabinet 120, to doors 128, or are otherwise positioned in view of fresh food chamber 122. As shown in FIG. 3, a camera 192 of camera assembly 190 is mounted to cabinet 120 at a front opening of fresh food chamber 122 and is oriented to have a field of view 194 directed across the front opening and/or into fresh food chamber 122.

Although a single camera 192 is illustrated in FIG. 3, it should be appreciated that camera assembly 190 may include a plurality of cameras 192 positioned within cabinet 120, wherein each of the plurality of cameras 192 has a specified monitoring zone or range positioned around fresh food chamber 122. In this regard, for example, the field of view 194 of each camera 192 may be limited to or focused on a specific area within fresh food chamber 122, such as one camera 192 for each drawer 140, one camera 192 for the bins 138 on the doors 128, etc.

Notably, however, it may be desirable to position each camera 192 proximate the front opening of fresh food chamber 122 and orient each camera 192 such that the field of view 194 is directed into fresh food chamber 122. In this manner, privacy concerns related to obtaining images of the user of the appliance 100 may be mitigated or avoided altogether. According to exemplary embodiments, camera assembly 190 may be used to facilitate an odor control process for refrigerator appliance 100. As such, each camera 192 may be positioned at an opening to fresh food chamber 122 to monitor food items that are being added to, removed from, and/or stored in fresh food chamber 122.

It should be appreciated that according to alternative embodiments, camera assembly 190 may include any suitable number, type, size, and configuration of camera(s) 192 for obtaining images of any suitable areas or regions within or around refrigerator appliance 100. In addition, it should be appreciated that each camera 192 may include features for adjusting the field of view and/or orientation.

It should be appreciated that the images obtained by camera assembly 190 may vary in number, frequency, angle, resolution, detail, etc. in order to improve the clarity of the particular regions surrounding or within refrigerator appliance 100. In addition, according to exemplary embodiments, controller 134 may be configured for illuminating the chilled chamber using one or more light sources prior to obtaining images. Notably, controller 134 of refrigerator appliance 100 (or any other suitable dedicated controller) may be communicatively coupled to camera assembly 190 and may be programmed or configured for analyzing the images obtained by camera assembly 190, e.g., in order to identify items being stored in, added to, and/or removed from refrigerator appliance 100, as described in more detail below.

In general, controller 134 may be operably coupled to camera assembly 190 for analyzing one or more images obtained by camera assembly 190 to extract useful information regarding objects located within the refrigerator appliance. In this regard, for example, images obtained by camera assembly 190 may be used to extract a barcode, identify a product, monitor the motion of the product, or obtain other product information related to object. Notably, this analysis may be performed locally (e.g., on controller 134) or may be transmitted to a remote server (e.g., in the “edge,” the “fog,” and/or in the “cloud,” as those of ordinary skill in the art will recognize as referring to a remote server or database in a distributed computing environment including at least one remote computing device in communication with the local controller 134) for analysis.

Such analysis may be intended to facilitate odor management, e.g., by identifying one or more potential odor sources in the fresh food chamber 122. Such odor sources may include, e.g., food items or other articles which may generate certain volatile organic compounds (“VOCs”) in sufficient quantity to create a user-perceptible odor. The “user” in the term “user-perceptible” odor is used with reference to an average adult human with no impairments to their sense of smell, e.g., a “user-perceptible” odor includes any odor which such user would readily detect or perceive when accessing the refrigerator appliance 100, such as standing in front of the refrigerator appliance 100 within arm's reach (about twenty four inches to about thirty inches or less) of the fresh food chamber 122 while at least one door 128 is open.

The images obtained by camera assembly 190 may include one or more still images, one or more video clips, or any other suitable type and number of images suitable for identification of food items or other potential odor sources. For example, such potential odor sources may include aging food items such as produce or meat items. Thus, image analysis may include identifying leafy greens based on, e.g., color and/or shape, and may further include detecting a potential odor source based on changes in the color, e.g., darkening, and/or shape, e.g., shrinking, of the leafy greens. In a similar example, meat items may be identified as a potential odor source based on the color thereof, such as a degree or shade of pink, grey, or other colors in the meat items. The foregoing examples regarding meats and vegetables may include identifying such food items as a potential odor source after the food items have been stored for a period of time in the refrigerator appliance 100, or may include identifying such food items as a potential odor source immediately upon the food items being placed in the refrigerator appliance 100, e.g., if the color is off when the food item or items is or are first placed in the refrigerator appliance 100. Additional exemplary items which may be recognized by the imaging system may include inherently pungent items such as onions or garlic, particularly cut or chopped onions or garlic, certain types of cheeses, or other similar items. As further examples, additional potential odor sources which may be identified by the imaging system include spills or stains.

Notably, camera assembly 190 may obtain images upon any suitable trigger, such as a time-based imaging schedule where camera assembly 190 periodically images and monitors the food storage chamber, e.g., fresh food chamber 122, or at least a portion thereof. According to still other embodiments, camera assembly 190 may periodically take low-resolution images until motion (such as opening, e.g., sliding forward, of one or both drawers 140 or opening of one or both doors 128, etc.) is detected (e.g., via image differentiation of low-resolution images), at which time one or more high-resolution images may be obtained. According to still other embodiments, refrigerator appliance 100 may include one or more motion sensors (e.g., optical, acoustic, electromagnetic, etc.) that are triggered when an object is being added to or removed from the fresh food chamber 122 (or any other compartment or chamber, e.g., any chilled chamber, in the refrigerator appliance 100), and camera assembly 190 may be operably coupled to such motion sensors to obtain images of the object during such movement.

It should be appreciated that the images obtained by camera assembly 190 may vary in number, frequency, angle, resolution, detail, etc. in order to improve the clarity of food items. In addition, according to exemplary embodiments, controller 134 may be configured for illuminating a refrigerator light (not shown) while obtaining an image or images. Other suitable imaging triggers are possible and within the scope of the present subject matter.

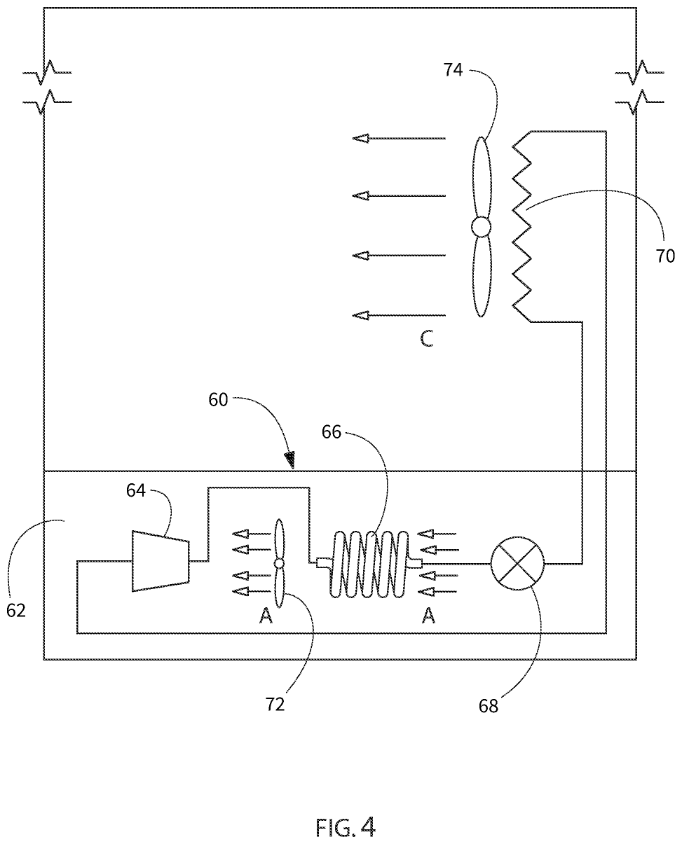

FIG. 4 provides a schematic view of the refrigerator appliance 100, in particular the sealed cooling system 60 thereof. As illustrated in FIG. 4, refrigerator appliance 100 includes a mechanical compartment 62 that at least partially contains components for executing a known vapor compression cycle for cooling air. The components include a compressor 64, a heat exchanger or condenser 66, an expansion device 68, and an evaporator 70 connected in series and charged with a refrigerant. Evaporator 70 is also a type of heat exchanger which transfers heat from air passing over the evaporator to a refrigerant flowing through evaporator 70 thereby causing the refrigerant to vaporize. As such, cooled air C is produced and configured to refrigerate chambers 122 and 124 of refrigerator appliance 100. The cooled air C may be directed to the food storage chambers 122 and 124 by a fan 74.

From evaporator 70, vaporized refrigerant flows to compressor 64, which operates to increase the pressure of the refrigerant. This compression of the refrigerant raises its temperature, which is lowered by passing the gaseous refrigerant through condenser 66 where heat exchange with ambient air takes place so as to cool the refrigerant. A fan 72 is used to pull air across condenser 66, as illustrated by arrows A, so as to provide forced convection for a more rapid and efficient heat exchange between the refrigerant and the ambient air.

Expansion device 68 further reduces the pressure of refrigerant leaving condenser 66 before being fed as a liquid to evaporator 70. Collectively, the vapor compression cycle components in a refrigeration circuit, associated fans, and associated compartments are sometimes referred to as a sealed refrigeration system operable to force cold air through refrigeration chambers 122 and 124. The refrigeration system 60 depicted in FIG. 4 is provided by way of example only. It is within the scope of the present invention for other configurations of the refrigeration system to be used as well. For example, fan 74 may be repositioned so as to push air across evaporator 70, dual evaporators may be used with one or more fans, and numerous other configurations may be applied as well.

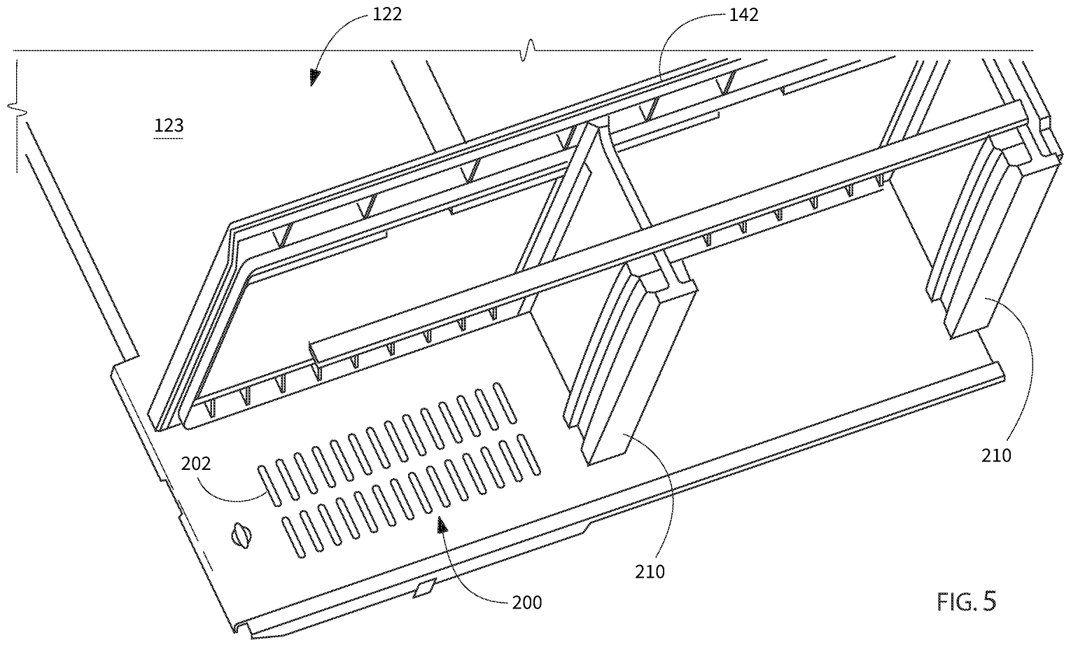

FIG. 5 illustrates an exemplary sub-assembly for a refrigerator appliance, such as the exemplary refrigerator appliance 100 described above. The sub-assembly may include an internal wall, e.g., a back wall 123. The back wall 123 may be an internal wall of the refrigerator appliance 100 and, as such, may define a portion of a food storage chamber therein such as the fresh food chamber 122. A storage component such as shelf 142 may be mounted to the back wall 123 and the sub-assembly may further include a plurality of walls 210, such that the storage drawers 140 (FIG. 3) may be mounted to the sub-assembly, e.g., slidably mounted to and between the walls 210. As illustrated in FIG. 5, the storage drawers 140 (FIG. 3) are removed from the sub-assembly, and a grille 200 formed in the back wall 123 may be seen with the drawers 140 removed. In various embodiments, the grille 200 may be formed in different locations on the back wall 123 and/or in a different internal wall of the fresh food chamber 122 (or another food storage chamber of the refrigerator appliance) as well as or instead of the illustrated location. The grille 200 includes a plurality of apertures 202 which may provide fluid communication between the food storage chamber, e.g., fresh food chamber 122, and other internal components of the refrigerator appliance 100. For example, thermal insulation, e.g., foam, may be provided between the internal walls such as back wall 123 and the cabinet 120, with ducts or other passageways or conduits also extending through the space, e.g., within and surrounded by the thermal insulation, between the internal walls and the cabinet 120, such as air ducts by which the cooled air C travels to the fresh food chamber 122 and enters the fresh food chamber 122 through the grille 200, e.g., through one or more of the apertures 202 thereof. Thus, for example, the grille 200 may provide or permit fluid communication between the sealed cooling system 60, in particular the evaporator 70, and the fresh food chamber 122, e.g., such that cooled air C flows into the fresh food chamber 122 from the evaporator 70 through the grille 200, such as the cooled air C may be urged through the grille 200 by the fan 74.

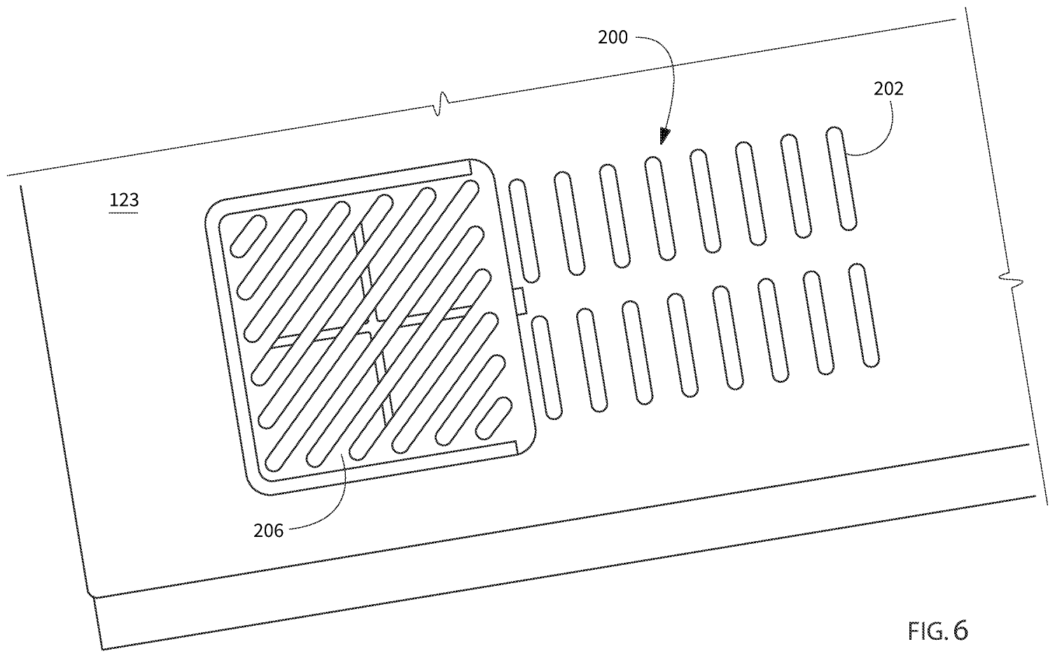

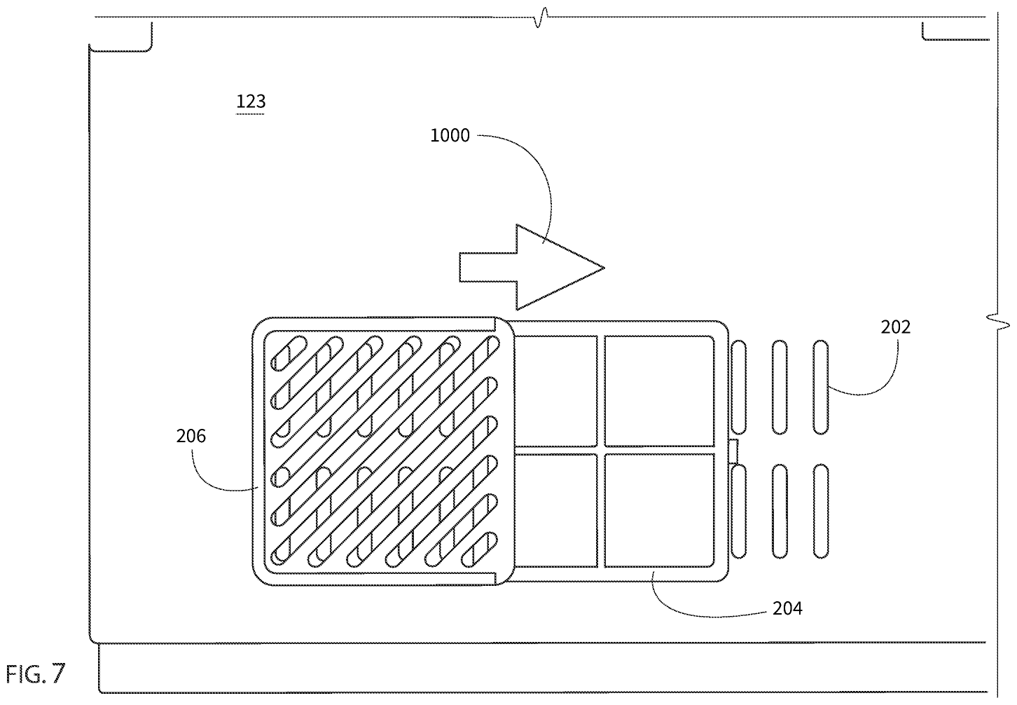

Turning now to FIGS. 6 and 7, the grille 200 may also include a filter cover 206. An air filter 204 (FIG. 7) may be removably received within the filter cover 206. Thus, when the air filter 204 is received within the filter cover 206 of the grille 200, the air filter 204 may thereby be positioned upstream of the fresh food chamber 122 with respect to the flow of cooled air C, such that air flowing into and/or circulating within the fresh food chamber 122 passes over and/or through the air filter 204, and the air filter 204 may thusly filter the air, e.g., reduce or remove odor-causing substances from the air flowing into and/or circulating within the fresh food chamber 122 (or other food storage chamber). As seen in FIG. 7 in particular, the air filter 204 may be slidable within and from the filter cover 206, e.g., along direction 1000 to remove the air filter 204, such as for maintenance, e.g., whereby the air filter 204 may be cleaned or replaced periodically as needed.

The air filter 204 may be any suitable air filter, e.g., may include one or more air filtration media, such as activated carbon, zeolite, or other similar media, including filter media with an additive or coating, such as a permanganate additive. For example, an activated carbon with a permanganate additive may advantageously promote increased adsorption of gasses having relatively smaller molecules as compared to activated carbon alone. In various embodiments, the air filter 204 may include granular activated carbon (GAC), agglomerated activated carbon (AAC), or other carbon filter media.

Using the teachings disclosed herein, one of skill in the art will understand that the present subject matter can be used with other types of refrigerators such as a refrigerator/freezer combination, side-by-side, bottom mount, compact, and any other style or model of refrigerator appliance. Accordingly, other configurations of refrigerator appliance 100 could be provided, it being understood that the configurations shown in the accompanying FIGS. and the description set forth herein are by way of example for illustrative purposes only. For example, description herein of detecting odor sources in fresh food chamber 122 are by way of example only, the present disclosure may also or instead be used to detect and/or identify potential odor sources in any portion of a refrigerator appliance.

Now that the construction and configuration of refrigerator appliance 100 and camera assembly 190 have been presented according to an exemplary embodiment of the present subject matter, exemplary methods for operating a refrigerator appliance, such as refrigerator appliance 100, are provided. Such methods may also be used to operate a camera assembly, e.g., camera assembly 190, or any other suitable camera assembly for monitoring appliance operation or inventory. In this regard, for example, controller 134 may be configured for implementing one or more of the following exemplary methods. However, it should be appreciated that the exemplary methods are discussed herein only to describe exemplary aspects of the present subject matter, and are not intended to be limiting.

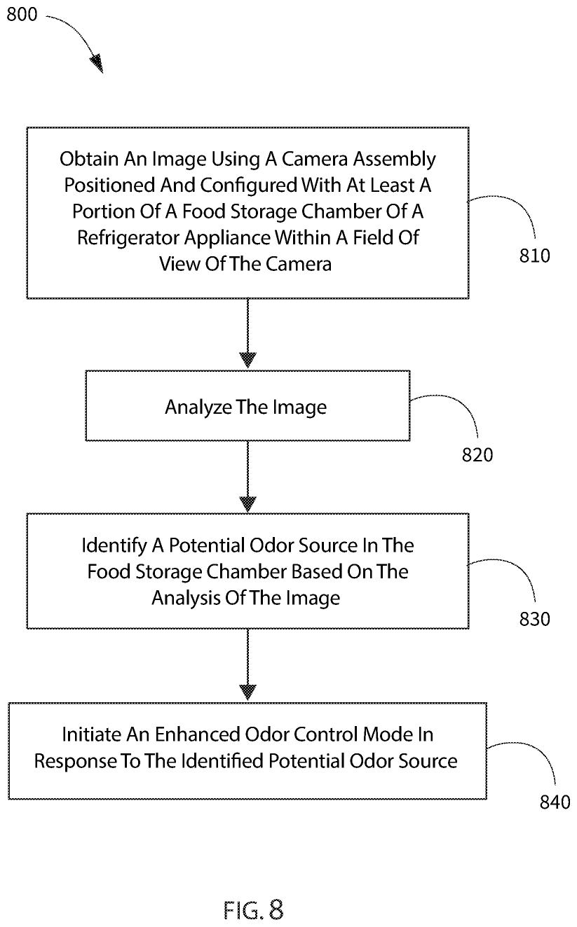

Turning now to FIG. 8, embodiments of the present disclosure may include a method 800 of operating a refrigerator appliance, such as the exemplary refrigerator appliance 100 described above. For example, the refrigerator appliance may include a cabinet defining a food storage chamber therein. Also by way of example, the refrigerator may further include a camera assembly positioned and configured with at least a portion of the food storage chamber within a field of view of the camera assembly, such as camera assembly 190 described above.

As shown in FIG. 8, method 800 includes, at step 810, obtaining an image using the camera assembly. For example, camera assembly 190 of refrigerator appliance 100 may obtain an image of areas within and/or around the refrigerator appliance 100, e.g., the image may include in its field of view at least a portion of the food storage chamber. In this regard, camera assembly 190 of refrigerator appliance 100 may obtain one or more images of fresh food chamber 122, freezer chamber 124, or any other zone or region within or around refrigerator appliance 100.

In the exemplary embodiment illustrated in FIG. 8, the method 800 may include a step 820 of analyzing the image that was obtained at step 810.

For example, controller 134 of refrigerator appliance 100 (or any other suitable dedicated controller) may be communicatively coupled to camera assembly 190 and may be programmed or configured for analyzing the images obtained by camera assembly 190, e.g., in order to identify potential odor sources in refrigerator appliance 100.

Step 820 includes analyzing the image, and such analysis may be used, for example, to identify objects, e.g., at least one potential odor source, which is or are disposed in the refrigerator appliance 100, e.g., in the fresh food chamber 122 or other food storage chamber. It should be appreciated that this analysis may utilize any suitable image analysis techniques, image decomposition, image segmentation, image processing, etc. This analysis may be performed entirely by controller 134, may be offloaded to a remote server for analysis, may be analyzed with user assistance (e.g., via user interface panel 136), or may be analyzed in any other suitable manner. According to exemplary embodiments of the present subject matter, the analysis performed at step 820 may include a machine learning image recognition process.

According to exemplary embodiments, this image analysis may use any suitable image processing technique, image recognition process, etc. As used herein, the terms “image analysis” and the like may be used generally to refer to any suitable method of observation, analysis, image decomposition, feature extraction, image classification, etc. of one or more images, videos, or other visual representations of an object. As explained in more detail below, this image analysis may include the implementation of image processing techniques, image recognition techniques, or any suitable combination thereof. In this regard, the image analysis may use any suitable image analysis software or algorithm to constantly or periodically monitor objects within the refrigerator appliance, such as within fresh food chamber 122 of the refrigerator appliance. It should be appreciated that this image analysis or processing may be performed locally (e.g., by controller 134) or remotely (e.g., by offloading image data to a remote server or other remote computing device, e.g., in the cloud).

Specifically, the analysis of the one or more images may include implementation an image processing algorithm. As used herein, the terms “image processing” and the like are generally intended to refer to any suitable methods or algorithms for analyzing images that do not rely on artificial intelligence or machine learning techniques (e.g., in contrast to the machine learning image recognition processes described below). For example, the image processing algorithm may rely on image differentiation, e.g., such as a pixel-by-pixel comparison of two sequential images. This comparison may help identify substantial differences between the sequentially obtained images, e.g., to identify movement, the presence of a particular object, the existence of a certain condition, a change in color and/or shape of an object, etc. For example, one or more reference images may be obtained when a particular condition exists, and these references images may be stored for future comparison with images obtained during appliance operation. Similarities and/or differences between the reference image and the obtained image may be used to extract useful information for improving appliance performance. For example, image differentiation may be used to determine when a pixel level motion metric passes a predetermined motion threshold.

The processing algorithm may further include measures for isolating or eliminating noise in the image comparison, e.g., due to image resolution, data transmission errors, inconsistent lighting, or other imaging errors. By eliminating such noise, the image processing algorithms may improve accurate object detection, avoid erroneous object detection, and isolate the important object, region, or pattern within an image. In addition, or alternatively, the image processing algorithms may use other suitable techniques for recognizing or identifying particular items or objects, such as edge matching, divide-and-conquer searching, greyscale matching, histograms of receptive field responses, or another suitable routine (e.g., executed at the controller 134 based on one or more captured images from one or more cameras). Other image processing techniques are possible and within the scope of the present subject matter.

In addition to the image processing techniques described above, the image analysis may include utilizing artificial intelligence (“AI”), such as a machine learning image recognition process, a neural network classification module, any other suitable artificial intelligence (AI) technique, and/or any other suitable image analysis techniques, examples of which will be described in more detail below. Moreover, each of the exemplary image analysis or evaluation processes described below may be used independently, collectively, or interchangeably to extract detailed information regarding the images being analyzed to facilitate performance of one or more methods described herein or to otherwise improve appliance operation. According to exemplary embodiments, any suitable number and combination of image processing, image recognition, or other image analysis techniques may be used to obtain an accurate analysis of the obtained images.

In this regard, the image recognition process may use any suitable artificial intelligence technique, for example, any suitable machine learning technique, or for example, any suitable deep learning technique. According to an exemplary embodiment, the image recognition process may include the implementation of a form of image recognition called region based convolutional neural network (“R-CNN”) image recognition. Generally speaking, R-CNN may include taking an input image and extracting region proposals that include a potential object or region of an image. In this regard, a “region proposal” may be one or more regions in an image that could belong to a particular object or may include adjacent regions that share common pixel characteristics. A convolutional neural network is then used to compute features from the region proposals and the extracted features will then be used to determine a classification for each particular region.

According to still other embodiments, an image segmentation process may be used along with the R-CNN image recognition. In general, image segmentation creates a pixel-based mask for each object in an image and provides a more detailed or granular understanding of the various objects within a given image. In this regard, instead of processing an entire image—i.e., a large collection of pixels, many of which might not contain useful information—image segmentation may involve dividing an image into segments (e.g., into groups of pixels containing similar attributes) that may be analyzed independently or in parallel to obtain a more detailed representation of the object or objects in an image. This may be referred to herein as “mask R-CNN” and the like, as opposed to a regular R-CNN architecture. For example, mask R-CNN may be based on fast R-CNN which is slightly different from R-CNN. For example, R-CNN first applies a convolutional neural network (“CNN”) having multiple convolutional layers (conv1 through convX, where “X” is the last convolutional layer, e.g., five convolutional layers, conv1 through conv5), and then allocates it to zone recommendations on the convX, e.g., conv5, property map instead of the initially split into zone recommendations. In addition, according to exemplary embodiments, standard CNN may be used to obtain, identify, or detect any other qualitative or quantitative data related to one or more objects or regions within the one or more images. In addition, a K-means algorithm may be used.

According to still other embodiments, the image recognition process may use any other suitable neural network process while remaining within the scope of the present subject matter. For example, the step of analyzing the one or more images may include using a deep belief network (“DBN”) image recognition process. A DBN image recognition process may generally include stacking many individual unsupervised networks that use each network's hidden layer as the input for the next layer. According to still other embodiments, the step of analyzing one or more images may include the implementation of a deep neural network (“DNN”) image recognition process, which generally includes the use of a neural network (computing systems inspired by the biological neural networks) with multiple layers between input and output. Other suitable image recognition processes, neural network processes, artificial intelligence analysis techniques, and combinations of the above described or other known methods may be used while remaining within the scope of the present subject matter.

In addition, it should be appreciated that various transfer techniques may be used but use of such techniques is not required. If using transfer techniques learning, a neural network architecture may be pretrained such as VGG16/VGG19/ResNet50 with a public dataset then the last layer may be retrained with an appliance specific dataset. In addition, or alternatively, the image recognition process may include detection of certain conditions based on comparison of initial conditions, may rely on image subtraction techniques, image stacking techniques, image concatenation, etc. For example, the subtracted image may be used to train a neural network with multiple classes for future comparison and image classification.

It should be appreciated that the machine learning image recognition models may be actively trained by the appliance with new images, may be supplied with training data from the manufacturer or from another remote source, or may be trained in any other suitable manner. For example, according to exemplary embodiments, this image recognition process relies at least in part on a neural network trained with a plurality of images of the appliance and/or contents thereof in different configurations, experiencing different conditions, or being interacted with in different manners. This training data may be stored locally or remotely and may be communicated to a remote server for training other appliances and models.

It should be appreciated that image processing and machine learning image recognition processes may be used together to facilitate improved image analysis, object detection, or to extract other useful qualitative or quantitative data or information from the one or more images that may be used to improve the operation or performance of the appliance. Indeed, the methods described herein may use any or all of these techniques interchangeably to improve image analysis process and facilitate improved appliance performance and consumer satisfaction. The image processing algorithms and machine learning image recognition processes described herein are only exemplary and are not intended to limit the scope of the present subject matter in any manner.

In some embodiments, the method may also include, and/or the refrigerator appliance may further be configured for, identifying one or more potential odor sources, e.g., based on analysis of the one or more images, as indicated at 830 in FIG. 8. In some embodiments, identification of the potential odor sources may be accomplished with the camera assembly 190. For example, the refrigerator appliance may include a camera, and the step of identifying the potential odor sources may include identifying the potential odor sources based on an image captured by the camera, such as in image of the potential odor sources or of an area including the potential odor sources as well as other objects. The structure and operation of cameras are understood by those of ordinary skill in the art and, as such, the camera is not illustrated or described in further detail herein for the sake of brevity and clarity. In such embodiments, the controller 134 of the refrigerator appliance 100 may be configured for image-based processing, e.g., to identify a potential odor source based on an image of the potential odor source, e.g., a photograph of or including the potential odor source(s) taken with the camera(s) 192 of the camera assembly 190. For example, the controller 134 may be configured to identify the potential odor sources by comparison of the image to a stored image of a known or previously-identified odor source. In additional embodiments, the image processing and/or analysis may be performed, in whole or in part, remotely. For example, in a distributed computing environment such as the “edge,” the “fog,” and/or in the “cloud,” as mentioned above.

In some embodiments, the step 830 may use the same image or multiple images from the same set of images, where the set of images includes multiple images of the same area or location taken over time. For example, the identification of the potential odor source may include image analysis whereby a change in color in a food item, such as darkening or turning brown, etc., of a fruit item, vegetable item, or other similar produce item is recognized from a chronological series of images of the same objects in the drawer.

Referring again to FIG. 8 in particular, the exemplary method 800 may further include a step 840 of initiating an enhanced odor control mode in response to the identified potential odor source. For example, the enhanced odor control mode may include activating a fan to circulate air within the food storage chamber. In some embodiments, the fan may also be paired with an air filter, whereby the fan is operable to and configured to urge air over, across, and/or through the air filter and thereby remove some or all of the odor from the air within the food storage chamber. For example, the air filter and the fan may be positioned proximate one another and upstream of the food storage chamber with respect to an air flow path within the refrigerator appliance, whereby activation of the fan causes the fan to urge air across the air filter and into the food storage chamber. For example, in embodiments where the air filter and the fan are both upstream of the food storage chamber, either the air filter or the fan may be upstream of the other. Thus, in some embodiments, the fan may urge the air across the air filter and into the food storage chamber by pulling the air across the air filter, i.e., the fan may be downstream of the air filter with respect to the air flow path within the refrigerator appliance. In additional embodiments, the fan may urge the air across the air filter and into the food storage chamber by pushing the air across the air filter, i.e., the fan may be upstream of the air filter with respect to the air flow path within the refrigerator appliance. Such variations may include variations in the relative positions of the air filter and the fan and/or variations in the direction of operation of the fan, such as a direction of rotation of the fan blades when activated or variations in the angle of the fan blades. Additionally, in such embodiments, the air filter may be immediately upstream of the fan or immediately downstream of the fan, or may be separated from the fan only by the internal wall of the food storage chamber and the grille in the wall.

In some embodiments, the fan may be a fan of a sealed cooling system, such as the evaporator fan 74 illustrated in FIG. 4 and described above. In such example embodiments, the enhanced odor control mode may include activating the fan at a higher level than during normal, cooling-driven, operation, such as at a faster rotational speed and/or longer on time duration than would be provided when the fan is operated solely based on the call for cooling of the food storage chamber. For example, in some embodiments where the refrigerator appliance includes a fan, the method may include operating the fan at a first level during a normal mode, where the first level may be determined by a call for cooling based on a set temperature for the food storage chamber, and the enhanced odor control mode may include operating the fan at a second level greater than the first level.

In embodiments where the enhanced odor control mode includes higher-level, e.g., faster rotational speed and/or longer on time, operation of the cooling fan as compared to the level of operation of the cooling fan based on the call for cooling, the enhanced odor control mode may thereby also include increased cooling of the food storage chamber, e.g., an increased amount and/or rate of cooled air C (FIG. 4) being directed into the food storage chamber than is necessary to reach the set temperature, which may result in cooling the food storage chamber to one or more temperatures less than the set temperature. In such instances, it may also be desirable to offset some of the increased cooling effect. For example, in some embodiments, the method may also include, after initiating the enhanced odor control mode, measuring a temperature within the food storage chamber, comparing the measured temperature with set temperature, and activating a heat source when the measured temperature is less than the set temperature. In various embodiments, the heat source may be a light, such as a light positioned and configured to illuminate the food storage chamber, e.g., when the door is opened, and/or a heater, such as an electric resistance heating element.

In embodiments where the fan is a fan of a sealed cooling system, such as the evaporator fan 74, and in instances when the enhanced odor control mode is activated or initiated while the cooling system, e.g., system 60, is not running, such as the compressor 64 is not motivating the refrigerant through the system 60, and in particular through the evaporator 70 thereof, then air that is introduced into and/or circulated within the food storage chamber while operating the fan at the second level greater than the first level may be at or about the same temperature as the remainder of the food storage chamber. Thus, when the cooling system is not running at the time that the enhanced odor control mode is initiated, the odor control mode is less likely to result in overcooling, such that the heat source may not be activated in such circumstances.

In some embodiments, identifying the potential odor source based on the analysis of the image may include estimating an age of one or more food items in the food storage chamber. For example, identifying the potential odor source may include estimating that a food item is overripe or expired.

In some embodiments, method 800 may further include providing a user notification, e.g., sending a user notification to a local user interface (e.g., on the refrigerator appliance) and/or a remote user interface device. The user notification may include an indication or identification of the potential odor source, particularly in embodiments where the potential odor source is an expired food item which may be unsafe for consumption, or a spill which may require cleaning up, and/or when other remedial or precautionary steps may be needed to address the odor or other effects of the potential odor source. For example, method 800 may also include providing one or more user notifications. Such notifications may be provided locally, e.g., on the user interface panel 136 of the refrigerator appliance 100, and/or remotely, such as on a remote device not directly physically attached or connected to the refrigerator appliance, e.g., a smartphone, smart home system, or other similar device. The user notification may include one or more of a visual notification, e.g., illuminating an indicator light or providing a text notification, and/or an audible notification, such as a chime or alert tone, etc.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method of operating a refrigerator appliance, the refrigerator appliance comprising a cabinet defining a food storage chamber therein and a camera assembly positioned and configured with at least a portion of the food storage chamber within a field of view of the camera assembly, the method comprising:
obtaining an image using the camera assembly;
analyzing the image;
identifying, based on the analysis of the image, a potential odor source in the food storage chamber; and
initiating an enhanced odor control mode in response to the identified potential odor source.

2. The method of claim 1, wherein identifying the potential odor source based on the analysis of the image comprises estimating an age of one or more food items in the food storage chamber.

3. The method of claim 2, further comprising sending a user notification comprising an identity of the one or more food items.

4. The method of claim 1, wherein identifying the potential odor source based on the analysis of the image comprises detecting a spill in the food storage chamber.

5. The method of claim 1, wherein the refrigerator appliance further includes a fan, the method further comprising operating the fan at a first level during a normal mode, the first level determined by a call for cooling based on a set temperature for the food storage chamber, and wherein the enhanced odor control mode comprises operating the fan at a second level greater than the first level.

6. The method of claim 1, further comprising, after initiating the enhanced odor control mode, measuring a temperature within the food storage chamber, comparing the measured temperature with set temperature, and activating a heat source when the measured temperature is less than the set temperature.

7. The method of claim 6, wherein the heat source comprises a light.

8. The method of claim 6, wherein the heat source comprises a heater.

9. The method of claim 1, wherein the refrigerator appliance further comprises a fan and an air filter proximate to the fan, the air filter and the fan upstream of the food storage chamber with respect to an air flow path within the refrigerator appliance, whereby the fan is operable to urge air across the air filter and into the food storage chamber, and wherein the enhanced odor control mode comprises activating the fan.

10. A refrigerator appliance, comprising:
a cabinet defining a food storage chamber therein;
a camera assembly positioned and configured with at least a portion of the food storage chamber within a field of view of the camera assembly; and
a controller, wherein the controller is configured for:
obtaining an image using the camera assembly;
analyzing the image;
identifying, based on the analysis of the image, a potential odor source in the food storage chamber; and
initiating an enhanced odor control mode in response to the identified potential odor source.

11. The refrigerator appliance of claim 10, wherein the controller is configured for identifying the potential odor source based on the analysis of the image by estimating an age of one or more food items in the food storage chamber.

12. The refrigerator appliance of claim 11, wherein the controller is further configured for sending a user notification comprising an identity of the one or more food items.

13. The refrigerator appliance of claim 10, wherein the controller is configured for identifying the potential odor source based on the analysis of the image by detecting a spill in the food storage chamber.

14. The refrigerator appliance of claim 10, further comprising a fan, wherein the controller is further configured for operating the fan at a first level during a normal mode, the first level determined by a call for cooling based on a set temperature for the food storage chamber, and wherein the enhanced odor control mode comprises operating the fan at a second level greater than the first level.

15. The refrigerator appliance of claim 10, wherein the controller is further configured for, after initiating the enhanced odor control mode, measuring a temperature within the food storage chamber, comparing the measured temperature with set temperature, and activating a heat source when the measured temperature is less than the set temperature.

16. The refrigerator appliance of claim 15, wherein the heat source comprises a light.

17. The refrigerator appliance of claim 15, wherein the heat source comprises a heater.

18. The refrigerator appliance of claim 10, further comprising a fan and an air filter proximate to the fan, the air filter and the fan upstream of the food storage chamber with respect to an air flow path within the refrigerator appliance, whereby the fan is operable to urge air across the air filter and into the food storage chamber, and wherein the enhanced odor control mode comprises activating the fan.
